(19) United States
(12) Patent Application Publication
(10) Pub. No.: US 2009/0325303 A1
(43) Pub. Date: Dec. 31, 2009

(54) PROCESS

(75) Inventor: Gary L. Corino, New South Wales (AU)

Correspondence Address: MERCHANT & GOULD PC, P.O. BOX 2903, MINNEAPOLIS, MN, 55402-0903, US

(73) Assignee: FERMISCAN AUSTRALIA PTY LIMITED, Sydney, NSW (AU)

(21) Appl. No.: 12/306,134

(22) PCT Filed: Jun. 27, 2007
(86) PCT No.: PCT/AU07/00879

§ 371 Date: Sep. 9, 2009

(30) Foreign Application Priority Data
Jun. 29, 2006 (AU) 2006903508
Jan. 19, 2007 (AU) 2007900272
Jan. 22, 2007 (AU) 2007900308

(51) Int. Cl. G01N 33/68

(52) U.S. Cl. 436/86

(57) ABSTRACT

A method for detecting a presence of an abnormal component in a keratin sample taken from a subject suffering from a pathological state comprising the steps of exposing the keratin sample to a swelling substance so as to penetrate the keratin sample thereby producing a derived chemical substance; obtaining data from the derived chemical substance; comparing the data obtained from the derived chemical substance with a second group of data contained in a reference database so as to identify the presence of the abnormal component in the keratin sample; whereby detection of the abnormal component is consistent with a presence of the pathological state in the subject.

A method for detecting a presence of an abnormal component in a keratin sample taken from a subject suffering from a pathological state comprising the steps of exposing the keratin sample to a swelling substance so as to penetrate the keratin sample thereby producing a derived chemical substance; obtaining data from the derived chemical substance; comparing the data obtained from the derived chemical substance with a second group of data contained in a reference database so as to identify the presence of the abnormal component in the keratin sample; whereby detection of the abnormal component is consistent with a presence of the pathological state in the subject.

### BACKGROUND

A variety of research to date has indicated the presence of abnormal changes in components of keratin samples, such as hair, taken from subjects, who are afflicted with a pathological state which can include cancer.

X-ray diffraction analysis has shown that subjects with a plurality of certain types of cancers (colon, breast and prostate) and other pathological states (Alzheimer's disease) produce hair samples that have abnormalities in them. The abnormalities are detectable using X-ray diffraction techniques and are consistent with the presence of the pathological state itself.

Whilst X-ray diffraction techniques can identify the presence of an abnormality in hair or other keratin samples, X-ray diffraction techniques are limited in that they do not reveal the nature of the abnormal component (i.e. whether the abnormality represents an inclusion of a chemical substance not normally present or rather a defect in the structure of a keratin fibril for example in the hair). A structural abnormality therefore can have the same proportion of chemical molecules present but differ structurally in geometric orientation of the molecules or their chemical bonding. Therefore, the pathological state can lead to structural changes in chemical bonding in the hair even though the chemical molecules forming the hair are in the same proportion as occurs in normal hair.

A variety of different causes as to the nature of chemical abnormalities present in the hair of persons with a pathological state have been conjectured. It has been suggested that an increase in specific metals normally found in hair may be associated with various pathological states.

Some researchers have conjectured that different types of growth factors can be found in increased proportions in the hair of cancer patients. Other researchers have suggested that rather than specific additions to normal hair such as increases in the content of certain metals present, that abnormal structures, for example a truncated form of keratin, can be present in hair taken from persons with certain pathological states such as breast cancer.

Irrespective of the truth of the above conjectures the structure of hair, contains a tightly bound cuticle surrounding a dense cortex so that it is often extremely difficult to chemically penetrate hair in an attempt to identify the presence of an abnormal component.

If the abnormal component whether structural or an atypical inclusion in hair taken from a subject with a pathological state, can be isolated, then the pathological state can be identified with greater specificity and sensitivity. Identification of the abnormal component means that additional information is then provided about the nature of the pathological state.

It is therefore seen that a problem sought to be solved by the present invention is the ability to chemically penetrate a hair sample so as to detect an abnormal component within the hair sample taken from a subject with a pathological state.

It is an object of the present invention to address or at least ameliorate some of the above disadvantages.

Notes

1. The term “comprising” (and grammatical variations thereof) is used in this specification in the inclusive sense of “having” or “including”, and not in the exclusive sense of “consisting only of”.

2. The above discussion of the prior art in the Background of the invention, is not an admission that any information discussed therein is citable prior art or part of the common general knowledge of persons skilled in the art in any country.

### BRIEF DESCRIPTION OF INVENTION

In one broad form of the invention there is provided a method for detecting a presence of an abnormal component in a keratin sample taken from a subject suffering from a pathological state comprising the steps of:

a) exposing the keratin sample to a swelling substance so as to penetrate the keratin sample thereby producing a derived chemical substance;

b) obtaining data from the derived chemical substance;

c) comparing the data obtained from the derived chemical substance with a second group of data contained in a reference database so as to identify the presence of the abnormal component in the keratin sample;

whereby detection of the abnormal component is consistent with a presence of the pathological state in the subject.

Preferably, the swelling substance includes formic acid.

Preferably, the second group of data is correlated with the presence of the pathological state in the subject.

Preferably, the second group of data is causatively associated with the presence of the pathological state in the subject.

Preferably, the swelling substance is selected from a plurality of different swelling substances.

Preferably, the keratin sample is selected from a plurality of different keratin samples.

Preferably, the second group of data is selected from a plurality of different groups of data.

Preferably, the derived data and the second group of data are analyzed using a plurality of different methods of comparison.

Preferably, in use, the keratin sample can be obtained and analyzed in association with at least one of a pharmacy, a test kit, the subject's home, a health care clinic and a testing laboratory or any convenient location (office, field or barn).

Preferably, the subject is selected from the group consisting of a human, and an animal.

### BRIEF DESCRIPTION OF DRAWINGS

FIG. 1 shows the application of a swelling substance to a keratin sample for use in diagnostic testing.

FIG. 2 shows a plurality of different chemical substances being applied in the alternative to a keratin sample.

FIG. 3 shows a plurality of different keratin samples taken from different subjects with different pathological states (or the same subject with several pathological states) or from a plurality of different mammalian species.

FIG. 4 shows a plurality of different methods of analysis being used to analyze data produced according to the first embodiment disclosed in FIG. 1.

FIG. 5 shows the method of analyzing a keratin sample being implemented in use, wherein a sample can be collected from a subject at a health care clinic, a collecting room or using a kit at a convenient location to the subject (home, field, barn, etc).

FIG. 6 illustrates results of SAXS analysis applied to samples treated in accordance with embodiments of the method of the present invention.

### DETAILED DESCRIPTION OF PREFERRED EMBODIMENTS

Embodiments of the present invention will now be described with reference to the accompanying drawings wherein:

Definitions:

An “animal” is defined as a living organism characterized by the capacity for voluntary motion, sensation and the ingestion of food such as plants and other animals, and which has a non-cellulose cell wall.

The plurality of different selections and forms pertaining to the invention as claimed include the selections and forms as appearing in the body of the specification.

“Mammalian species” includes the types of species as appearing in the body of the specification. It can include a human, a pet such as a dog or cat or a variety of other animals with hair.

A “keratin sample” or “keratin substance” is a sample that is substantially comprised of keratin. The keratin substance 16 can include human scalp or body hair and in particular pubic hair, pet hair, animal hair or hair from a member of a mammalian species in general, or other keratin based materials such as nail clippings or an eyelash.

A “subject” can include a mammalian species. A mammalian species can include a human, a pet such as a dog or cat or a variety of other animals.

Unless otherwise indicated by the context, a claim to one element is consistent with a claim to at least one element.

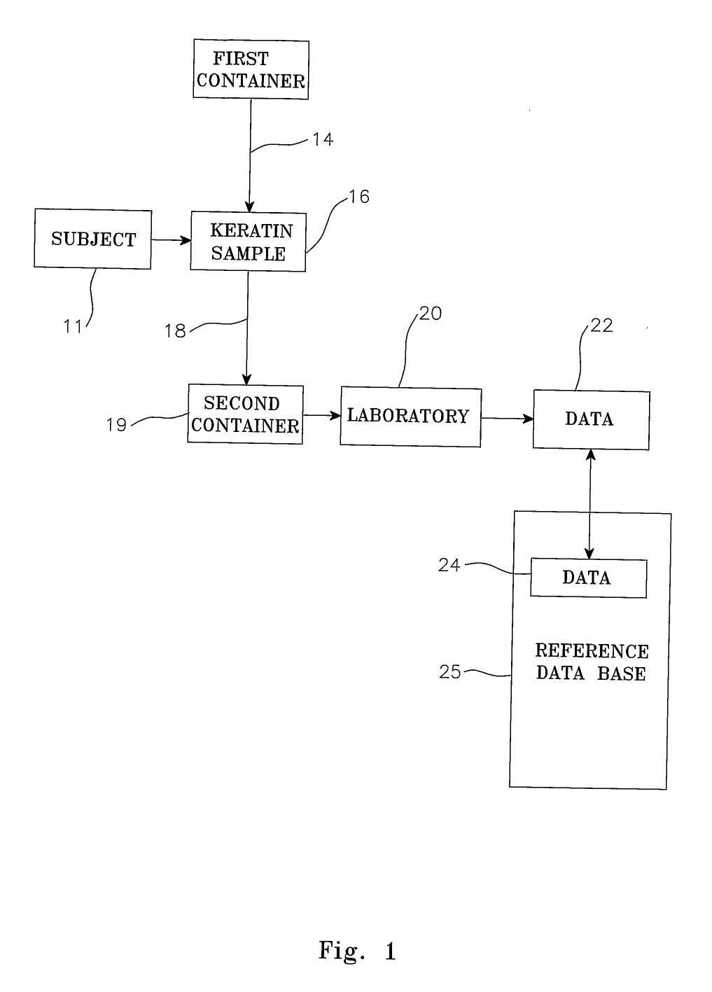

FIG. 1 illustrates a method of analyzing a keratin sample 16. FIG. 1 shows a first container 12 which holds a swelling substance 14. A keratin sample 16 is taken from subject 11, wherein the subject 11 can include a mammalian species. A mammalian species can include a human, a pet such as a dog or cat or a variety of other animals. The keratin substance 16 can include human scalp or body hair and in particular pubic hair, pet hair, animal hair or hair from a mammalian species in general, or other keratin based materials such as nail clippings or an eyelash.

The keratin sample 16 is exposed to the swelling substance 14. A derived chemical substance 18 is obtained from application of the swelling substance 14 to the keratin sample 16 whereby the swelling substance 14 penetrates the keratin sample 16. The derived chemical substance 18 is collected in the second container 19 (alternatively and without loss of generality only one container need be used).

The derived chemical substance 18 located in the second container 19 is then taken to a laboratory 20 at the place of application of the swelling substance 14. The laboratory 20 will then use a plurality of diagnostic tests to obtain data 22 which can be compared with data 24 located in a reference database 25 so as to determine if the subject 11 has a pathological state. The reference database 25 can be obtained from a plurality of control samples taken from normal subjects and subjects with a plurality of different pathological states.

The data 22 can be compared with data 24 in the reference database 25 to determine whether or not the subject 11 has a pathological state, for example if the reference database 25 indicates that the result in question is both correlated and causatively linked to a pathological state then a meaningful comparison can be considered. Additionally zero correlation or no information being provided in the case of no association with an abnormal component being present in the derived chemical substance 18 can in certain instances be consistent with an abnormality being a defect in protein folding, that is a structural defect in the keratin sample 16 as opposed to an additional inclusion of a component that may or may not be ordinarily found in the keratin sample 16 such as a metal or other compound (whereby no inclusion is washed out into the derived chemical substance 18).

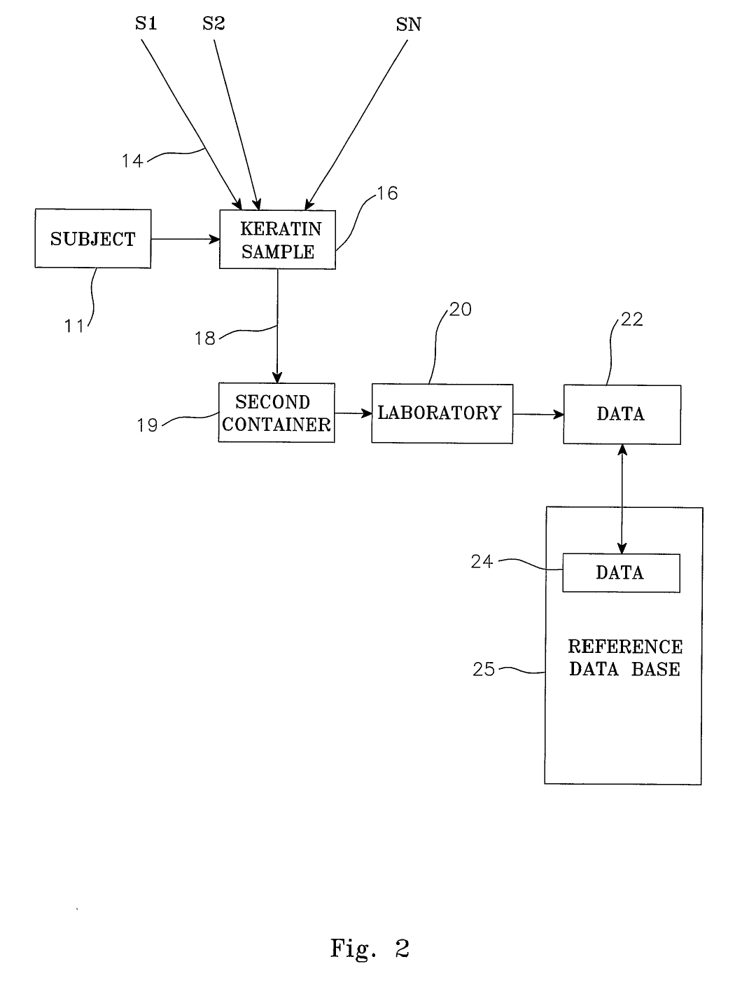

FIG. 2 shows an embodiment of the present invention in which the sensitivity or specificity of the method described in FIG. 1 is improved by way of changing the swelling substance 14. FIG. 2 shows a plurality of different swelling substances 14, being S1, S2 . . . SN.

In FIG. 2 the data 22, not shown, is analyzed by comparing the data 22 with the data 24 (from a reference database 25 as seen in FIG. 1) so as to select a swelling substance from the set S1 . . . SN, which is adapted to remove the defect in the keratin sample 16 by either altering the structural nature of the keratin sample 16 so as to restore normal folding and configuration of the keratin sample 16 or by transferring, by way of application of the swelling substance 14 to the keratin sample 16, the component causing the abnormality in the keratin sample 16 to the derived chemical substance 18 (this can involve for example formic acid penetrating and washing out an inclusion from a hair sample). Whilst formic acid has been disclosed herein a number of other substances are amenable to penetrate a keratin substance so as to remove an abnormal component; other substances include acetic acid, propionic acid, butyric acid, trifluoracetic acid, monochloracetic acid, hydrochloric acid, dimethyl formamide, potassium iodide, sodium bromide, lithium bromide, lithium chloride, DTT (Dithio-Threitol) or DTE (Dithioerythritol) and urea or other protein denaturing agents. However, unlike formic acid other considerations are relevant such as toxicity and in the case of reducing agents the fact that the structure of the keratin sample 16 can be substantially destroyed by the application of the reducing agent. In contrast formic acid is a swelling substance 14 which may not destroy the structure of a keratin sample 16 after application.

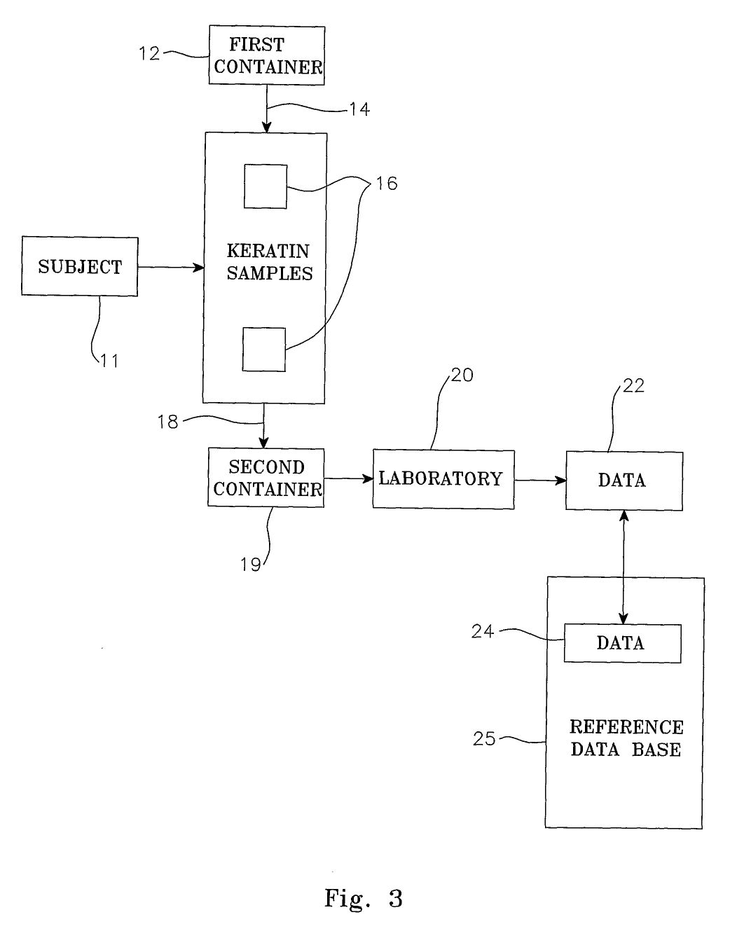

FIG. 3 shows the use of a specific swelling substance 14, for a given mode of operation in association with a plurality of different keratin samples 16 to yield a derived chemical substance 18. The plurality of different keratin samples 16 can be taken from a plurality of different mammalian species. Alternatively or additionally, the plurality of different keratin samples 16 can be taken from subjects 11 who are suspected to have a plurality of different pathological states.

The variations in swelling substances as disclosed in FIG. 2 and the variations in keratin samples 16 as disclosed in FIG. 3 (two are shown but more can be envisaged) can be used to identify a particular capability in a particular swelling substance 14 or a particular susceptibility in a given keratin sample 16. For example if a given swelling substance 14 such as formic acid was known to remove an abnormal component from a keratin sample 16 during a particular concentration range and a particular sub-range of the concentration was shown to yield a significantly improved result then a new availability in the formic acid could exist.

Similarly, as seen in FIG. 3, for a given swelling substance 14, having a particular concentration, a systematic variation in the types of keratin samples 16 could disclose a susceptibility of a particular type of keratin sample 16 to a swelling substance 14 such as formic acid.

A particular susceptibility will occur if a keratin sample 16 is known to yield a conclusive result in association with a given mode of operation and a given swelling substance 14 that leads to an improvement in specificity or sensitivity of a diagnostic test to look for the presence of the abnormal component.

The plurality of different keratin samples 16, shown in FIG. 3 can include a keratin sample 16 taken from a subject 11 who is suspected of having a pathological state which can include one or more cancers or pathological states such as lung cancer, Creutzfeldt-Jacob disease, mad cow disease, infection (bacterial, viral, or a Prion or more generally by other agents), a metabolic disorder (which can include diabetes), or alternatively hepatitis, heart disease or liver dysfunction. Further, without limitation the keratin sample 16 can include Keratin Type I and Keratin Type II.

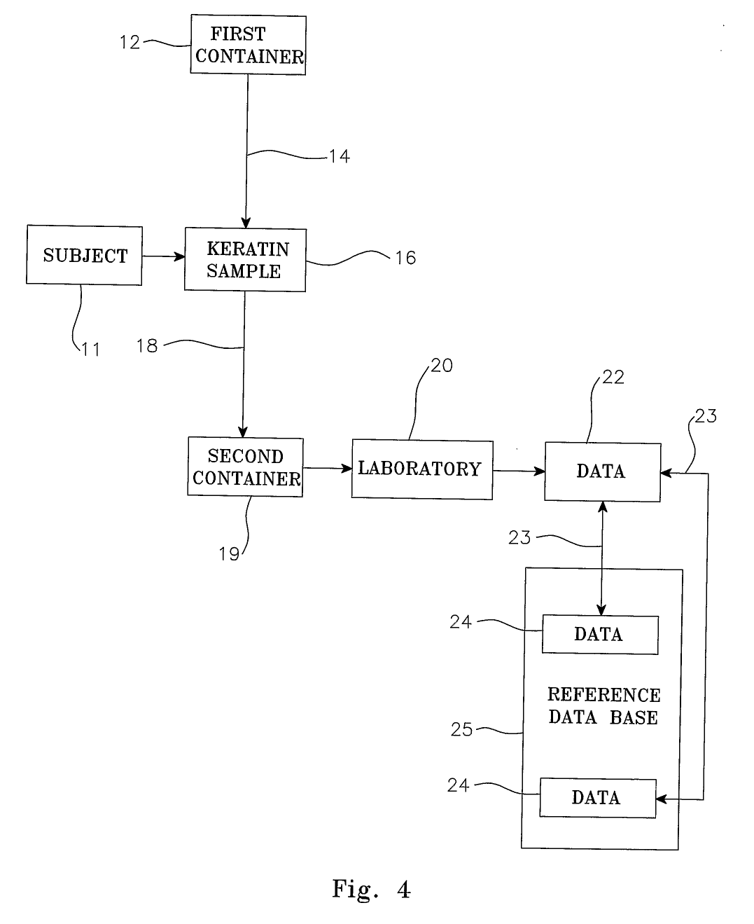

FIG. 4 shows an embodiment of the method for detecting the presence of a pathological state in which a given swelling substance 14 and a given keratin sample 16 are used in association with a plurality of different types of methods of comparison between the data 22 and a second group of data 24 contained in the reference database 25 so as to produce an improvement in specificity or sensitivity of the method of analyzing the keratin sample 16. The plurality of different comparison's 23 shown in FIG. 4 (two are shown but more can be envisaged) can without limitation include variations in the mode of operation of the method of analyzing a keratin sample including spectral analysis or the use of pattern recognition computer programs.

An advantage of the present embodiment is that if the abnormal component is present in the derived chemical substance 18 then the derived chemical substance 18 can be further analyzed for the purpose of identifying the nature of the abnormal component so as to provide a health care practitioner with more information about the pathological state. If the abnormal component is a trace metal then atomic absorption spectroscopy or ICP-mass spectrometry could be applied to the derived chemical substance 18 to confirm the nature of the abnormal component. If the abnormal component in the derived chemical substance 18 is a protein, a carbohydrate, a fatty acid or more generally of organic origin and an antibody can be raised against the abnormal component then a plurality of different techniques such as Western blot analysis, ELISA or cell agglutination assays can be used in an attempt to characterize the abnormal component. In certain instances, immuno-electron microscopy can be used in an attempt to identify the abnormal component in the keratin sample 16 without the need for the use of the derived chemical substance 18 to subsequently be obtained. If the abnormal component is associated with genetic material (being hereditary in nature or acquired by way of a viral vector) then an amplification technique could be applied to determine the sequence and conformation of the abnormal component.

In Use

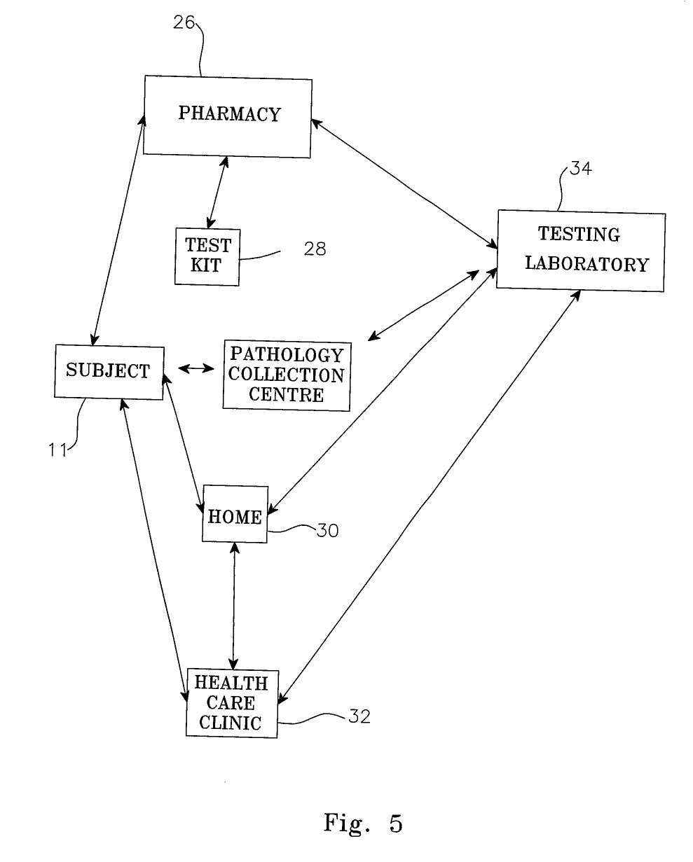

FIG. 5 shows an embodiment of the present invention in use. In FIG. 5 a keratin sample 16 can be collected from a subject at a pharmacy 26. The keratin sample 16 can then be sent to a testing laboratory 34 so as to perform the method for detecting the presence of an abnormal component in the keratin sample 16 as seen in FIG. 1.

A test kit 28 can be obtained so as to use the test kit 28 embodying the method of detection at the subject's home 30, in association with consultation of the subject's health care practitioner located at a health care clinic 32.

Alternatively, without the need of a home test the subject 11 can visit his or her health care clinic 32 so as to provide the keratin sample 16. The health care clinic 32 can perform the method of analyzing the keratin sample 16 themselves or in a further, preferred embodiment the health care clinic 32 can obtain the keratin sample 16 from the subject 11 so as to forward the keratin sample 16 to the testing laboratory 34.

Whilst the embodiments above have been restricted to members of a mammalian species, it is conceivable that any animal which exhibits keratin changes that are causatively linked to the presence of pathology can be the subject of testing using a method according to the present invention.

Further Embodiment

Organic Acid Treatment of Hair Fibers

It is known that immersion of hair fibers in organic acids such as formic acid and acetic acid can cause them to swell in diameter by as much as 50% and this effect can be reversed by rinsing in water. Blackburn and Lowther (Blackburn S and Lowther A G. The action of organic acids on some fibrous proteins: the oxidation of wool keratin. Biochem J. 1951; 49:554-9) reported that a small amount of protein could be extracted from wool fibers by treatment in either formic or acetic acid at room temperature. This methodology was applied to several hair fibers from individuals of known pathology to determine if the ring observed by Synchrotron Small Angle X-Ray Scatter (SAXS) of hair, specific for the presence of breast cancer, could be removed by such treatment. Hair fibers from subjects with breast cancer were immersed in an 85% (v/v) solution of formic acid or in glacial acetic acid for 3 minutes at room temperature. The acid was then decanted and replaced with several changes of Milli-Q water. The fibers were allowed to dry then mounted into a sample holder for exposure to an X-ray source. SAXS image data was collected for these hairs.

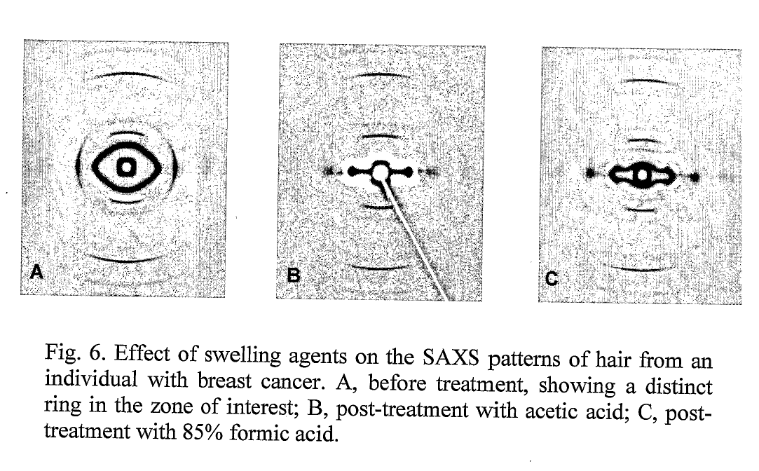

Hair fibers that were taken from the same individual and subsequently treated with formic and acetic acid showed either significant reduction or complete removal of the ring in the zone of interest in the SAXS image. A typical example is demonstrated in FIG. 6. FIG. 6A is a SAXS image of a hair from an individual with breast cancer. FIGS. 6B and 6C are SAXS images of hair fibers from the same individual post treatment with acetic acid and formic acid respectively. It can be seen from these images that the ring in the zone of interest is significantly diminished after treatment with either acid and hence such treatments may be used as a tool to investigate the underlying change in the fiber associated with the presence of breast cancer.

1. A method for detecting a presence of an abnormal component in a keratin sample taken from a subject suffering from a pathological state comprising the steps of:
a) exposing the keratin sample to a swelling substance so as to penetrate the keratin sample thereby producing a derived biological substance which is associated with an altered x-ray diffraction pattern in hair from subjects with a diseased state; said, swelling substance including organic acids known to swell wool and hair fibres, in particular formic acid and acetic acid;
b) obtaining data from the derived biological substance;
c) comparing the data obtained from the derived biological substance with a second group of data contained in a reference database so as to identify the presence of the abnormal component in the keratin sample; and
whereby detection of the abnormal component is consistent with a presence of the pathological state in the subject.

2. The method for detecting the presence of an abnormal component in a keratin sample as recited in claim 1 wherein the swelling substance includes formic acid.

3. The method for detecting the presence of an abnormal component in a keratin sample as recited in claim 1 wherein the second group of data is correlated with the presence of the pathological state in the subject.

4. The method for detecting the presence of an abnormal component in a keratin sample as recited in claim 1 wherein the second group of data is causatively associated with the presence of the pathological state in the subject.

5. The method for detecting the presence of an abnormal component in a keratin sample as recited in claim 1 wherein the swelling substance is selected from a plurality of different swelling substances.

6. The method for detecting the presence of an abnormal component in a keratin sample as recited in claim 1 wherein the keratin sample is selected from a plurality of different keratin samples.

7. The method for detecting the presence of an abnormal component in a keratin sample as recited in claim 1 wherein the second group of data is selected from a plurality of different data groups of data.

8. The method for detecting the presence of an abnormal component in a keratin sample as recited in claim 1 wherein the derived data and the second group of data are analyzed using a plurality of different methods of comparison.

9. The method for detecting the presence of an abnormal component in a keratin sample as recited in claim 1 wherein, in use, the keratin sample can be obtained and analyzed in association with at least one of a pharmacy, a test kit, the subject's home, a health care clinic and a testing laboratory.

10. (canceled)

11. The method for detecting the presence of an abnormal component in a keratin sample according to claim 1 wherein the subject is selected from the group consisting of a human, and an animal.
